(19) United States
(12) Patent Application Publication
(10) Pub. No.: US 2019/0117535 A1
(43) Pub. Date: Apr. 25, 2019

(54) ORAL RINSE

(71) Applicant: Church & Dwight Co., Inc., Princeton, NJ (US)

(72) Inventors: Anthony J. Cirigliano, Chesterfield, NJ (US); Beth Galinskie, Brick, NJ (US); Jennifer Benson, Newtown, PA (US); Bruce Strasser, Lawrenceville, NJ (US)

(73) Assignee: Church & Dwight Co., Inc., Princeton, NJ (US)

(21) Appl. No.: 15/789,716

(22) Filed: Oct. 20, 2017

(51) Int. Cl. A61K 8/27; A61Q 11/00; A61K 8/34; A61K 8/24

(52) U.S. Cl. CPC A61K 8/27; A61Q 11/00; A61K 2800/92; A61K 8/24; A61K 8/345

(57) ABSTRACT

The present invention provides an aqueous oral rinse composition which includes zinc chloride, sorbitol, one or more pyrophosphates, and water. Methods of providing such an oral rinse composition are also provided herein.

### FIELD OF THE INVENTION

The present invention relates to oral compositions useful for providing pain relief, and more particularly to an aqueous-based oral rinse composition.

### BACKGROUND

This invention relates to a process of including zinc chloride into an aqueous-based oral rinse composition using a sugar alcohol to improve zinc chloride solubility and thereby prevent or at least reduce formation of a salty precipitate from forming that is insoluble in the aqueous base. The present invention further relates to oral rinse compositions produced according to methods disclosed herein.

It is well known in the art that adding zinc chloride to excess water results in the formation of zinc oxychloride. The presence of zinc salts in oral compositions can result in an unfavorable aftertaste, which is not preferred by users. As such, currently available oral rinse compositions are not aqueous-based and/or require additional ingredients useful in masking the unpleasant aftertaste associated with the presence of undesirable zinc salts in the oral rinse composition.

Accordingly, there is still a desire and a need to provide an aqueous-based oral rinse composition comprising zinc chloride as an active ingredient without the presence of zinc salts. It is further desirable to provide a method of forming an aqueous-based oral rinse composition comprising zinc chloride and having substantially no zinc salts precipitate in the final composition.

### SUMMARY OF THE INVENTION

In one aspect of the present invention, an oral rinse composition comprising: zinc chloride; water, wherein the amount of water present in the composition is at least about 50%, or at least about 80%, based on the total weight of the composition; one or more sugar alcohols; and one or more pyrophosphates. In various embodiments, the oral rinse composition is substantially free of one or both of zinc oxychloride and zinc phosphate.

In some embodiments of the oral rinse compositions described herein, the zinc chloride is present in an amount in the range of about 0.01 to about 5 weight percent, based on the total weight of the oral rinse composition. In various embodiments, the one or more pyrophosphates are present in amounts in the range of about 0.01 to about 5 weight percent, based on the total weight of the oral rinse composition. In certain embodiments, the one or more sugar alcohols are present in amounts in the range of about 15 to about 35 weight percent, based on the total weight of the oral rinse composition.

In various embodiments of the oral rinse compositions described herein, the one or more pyrophosphates comprise at least one of tetrasodium pyrophosphate (TSPP) and disodium pyrophosphate (DSPP). In some embodiments, the one or more sugar alcohols comprise sorbitol.

A method of making an oral rinse composition is also provided herein. In various embodiments, the method comprises:providing a concentrated zinc chloride solution; forming a premix comprising the zinc chloride solution, one or more sugar alcohols, and deionized water; mixing the premix with one or more pyrophosphates until the pyrophosphates are dissolved in an intermediate solution; and mixing additional ingredients into the intermediate solution to form the oral rinse composition. In certain embodiments, the oral rinse compositions provided according to methods described herein are substantially free of zinc oxychloride and zinc phosphate.

In some embodiments of the methods described herein, the zinc chloride solution is a 50% zinc chloride in water solution. In certain embodiments, the amount of the deionized water present in the zinc chloride solution is no more than 65%, based on the total weight of the zinc chloride solution.

In various embodiments, the method of making an oral rinse composition further comprises reducing the pH of the premix to a pH in the range of about 5-7. In certain embodiments, the method further comprises increasing the temperature of the water used to form the zinc chloride solution before adding the zinc chloride or heating the zinc chloride solution to help the zinc chloride fully dissolve.

In various embodiments, the oral rinse compositions described herein can be manufactured in the form of a final oral rinse product having a desired dosage size, shape and weight.

Other aspects and advantages of the present invention will become apparent from the following.

### DETAILED DESCRIPTION OF THE INVENTION

The present disclosure now will be described more fully hereinafter. The disclosure may be embodied in many different forms and should not be construed as limited to the embodiments set forth herein; rather, these embodiments are provided so that this disclosure will satisfy applicable legal requirements. Like numbers refer to like elements throughout. As used in this specification and the claims, the singular forms “a,” “an,” and “the” include plural references unless the context clearly dictates otherwise.

In one aspect of the present invention, an oral rinse composition is provided comprising zinc chloride, at least one sugar alcohol, at least one pyrophosphate, and water. In certain embodiments, the oral rinse composition further includes additional ingredients, as discussed in more detail below. The main advantages of the invention include a providing an aqueous oral rinse composition that can be substantially free of one or both of zinc oxychloride and zinc phosphate beyond a trace amount. A trace amount of zinc oxychloride and/or zinc phosphate (separately or in combination) may be considered to be less than 0.1 weight percent or less than 0.01 weight percent based on the total weight of the oral rinse composition. Accordingly, via methods disclosed herein, zinc chloride powder can be included into an aqueous oral rinse composition without forming a precipitate zinc salt in a significant amount.

In various embodiments of the present invention, the oral rinse composition can be an aqueous composition in the form of a liquid. In some embodiments, deionized water can be present in an amount in the range of about 50 to about 85 weight percent, about 60 to about 80 weight percent, or about 65 to about 75 weight percent, based on the total weight of the oral rinse composition. In various embodiments, deionized water can be present in an amount of at least about 50 weight percent, at least about 60 weight percent, or at least about 65 weight percent, based on the total weight of the oral rinse composition (e.g., with a maximum concentration of about 85 weight percent) based on the total weight of the oral rinse composition. It is noted that this reference to deionized water in the oral rinse compositions described herein does not include additional water which may be present in the oral rinse composition in the form of a solution. As discussed in more detail below, the oral rinse compositions described herein can further include a concentrated zinc chloride solution and a sugar alcohol solution. As such, water, which includes the water present in the various solutions which make up the final oral rinse composition, can be present in an amount in the range of about 90 to about 99 weight percent, or about 95 to about 98 weight percent based on the total weight of the oral rinse composition.

In various embodiments, one or more active ingredients can be included in the oral rinse composition to provide astringency, pain relief and/or soothing and cooling effects of the product. The one or more active ingredients can be present in an amount suitable for providing oral pain relief. For example, zinc chloride can be included in the oral rinse composition as an active ingredient for astringency. In various embodiments, the zinc chloride can be in the oral rinse composition in the form of a zinc chloride concentrate (i.e., a concentrated zinc chloride solution). For example, the concentrated zinc chloride solution can be about 40% to about 75%, or about 45% to about 70%, or about 50% to about 65% zinc chloride in water. The zinc chloride can be present in an amount of, for example, about 0.01 to about 3.0, or about 0.1 to about 1 weight percent, or about 0.1 to about 0.5 weight percent, based on the total weight of the oral rinse composition. In certain embodiments, a concentrated zinc chloride solution (e.g., a 50-65% zinc chloride solution) can be present in the oral rinse compositions described herein in an amount in the range of about 0.01 to about 10 weight percent, or about 0.1 to about 5 weight percent, based on the total weight of the oral rinse composition. In various embodiments, menthol can be used in the oral rinse composition to provide additional pain relief specifically to the gums via the cooling effect of the active ingredient. Menthol can be present in an amount of, for example, about 0.01 to about 3 weight percent, based on the total weight of the oral rinse composition. Other active ingredients known in the art can be added to the oral rinse product as desired. Moreover, the total amount of active ingredient(s) present in the oral rinse composition can vary based on the type of active ingredient(s) used and/or the number of different active ingredients used.

In various embodiments, the oral rinse composition can further comprise one or more sugar alcohols. For example, the oral rinse composition can comprise sorbitol. In certain embodiments, the sugar alcohol can be provided in the form of a sugar alcohol solution. For example, the oral rinse composition can comprise a 50-80% sugar alcohol solution. In some embodiments, a sugar alcohol solution can be present in an amount in the range of about 0.1 to about 40 weight percent, about 5 to about 35 weight percent, or about 15 to about 30 weight percent, based on the total weight of the oral rinse composition. In various embodiments, a sugar alcohol solution can be present in an amount of at least about 0.01 weight percent, at least about 5 weight percent, or at least about 15 weight percent, based on the total weight of the oral rinse composition (e.g., with a maximum concentration of about 40 weight percent). In certain preferred embodiments, a 70% sugar alcohol can be present in an amount in the range of about 5 to about 40 weight percent, or about 15 to about 35 weight percent, based on the total weight of the oral rinse composition.

In various embodiments, the oral rinse composition can further comprise one or more pyrophosphates. In some embodiments, the one or more pyrophosphates is selected from the group consisting of tetrasodium pyrophosphate (TSPP), disodium pyrophosphate (DSPP), and combinations thereof. In some embodiments, a pyrophosphate can be present in an amount in the range of about 0.01 to about 5 weight percent, about 0.1 to about 3 weight percent, or about 0.1 to about 2 weight percent, based on the total weight of the oral rinse composition. In various embodiments, a pyrophosphate can be present in an amount of at least about 0.01 weight percent, at least about 0.1 weight percent, or at least about 0.5 weight percent, based on the total weight of the oral rinse composition (e.g., with a maximum concentration of about 5 weight percent).

In various embodiments, the oral rinse composition can further comprise at least one alcohol. In certain embodiments, the oral rinse composition can comprise ethyl alcohol. In certain embodiments, the at least one alcohol can be present in an amount in the range of about 1 to about 5 weight percent, or about 2 to about 3 weight percent, based on the total weight of the oral rinse composition. In certain embodiments, the one or more alcohols can be present in an amount of at least about 1 weight percent, or at least about 2 weight percent, based on the total weight of the oral rinse composition. In some embodiments, it can be desirable to limit the amount of alcohol that is present in the composition. For example, the oral rinse composition may comprise no greater than 10 weight percent, no greater than 5 weight percent, or no greater than 2 weight percent of an alcohol. In some embodiments, the oral rinse composition can be substantially free of a non sugar alcohol (e.g., ethyl alcohol) beyond a trace amount (e.g., less than 0.1 weight percent or less than 0.01 weight percent based on the total weight of the oral rinse composition).

In various embodiments, the oral rinse composition can comprise benzyl alcohol. In certain embodiments, benzyl alcohol can be present in an amount in the range of about 0.01 to about 5 weight percent, or about 0.1 to about 1 weight percent, based on the total weight of the oral rinse composition. In certain embodiments, benzyl alcohol can be present in an amount of at least about 0.01 weight percent, or at least about 0.1, or at least about 0.2 weight percent, based on the total weight of the oral rinse composition. In some embodiments, it can be desirable to limit the amount of benzyl alcohol that is present in the composition. For example, the oral rinse composition may comprise no greater than 2 weight percent, no greater than 1 weight percent, or no greater than 0.5 weight percent of benzyl alcohol, based on the total weight of the oral rinse composition. In some embodiments, the oral rinse composition can be substantially free of benzyl alcohol beyond a trace amount (e.g., less than 0.1 weight percent or less than 0.01 weight percent based on the total weight of the oral rinse composition).

In various embodiments, the oral rinse composition can further comprise one or more emulsifiers. For example, the oral rinse composition can comprise polysorbate. In some embodiments, an emulsifier can be present in an amount in the range of about 0.01 to about 5 weight percent, or about 0.1 to about 1 weight percent, based on the total weight of the oral rinse composition. In various embodiments, an emulsifier can be present in an amount of at least about 0.01 weight percent, based on the total weight of the oral rinse composition (e.g., with a maximum concentration of about 5 weight percent).

In various embodiments, the oral rinse composition can further comprise one or more surfactants. For example, the oral rinse composition can comprise poloxamer. In some embodiments, a surfactant can be present in an amount in the range of about 0.01 to about 10 weight percent, about 0.1 to about 5 weight percent, or about 0.1 to about 3 weight percent, based on the total weight of the oral rinse composition. In various embodiments, a surfactant can be present in an amount of at least about 0.01 weight percent, based on the total weight of the oral rinse composition (e.g., with a maximum concentration of about 10 weight percent).

In various embodiments, the oral rinse composition can further comprise one or more preservatives. For example, the oral rinse composition can comprise sodium benzoate. In some embodiments, a preservative can be present in an amount in the range of about 0.01 to about 5 weight percent, about 0.01 to about 1 weight percent, or about 0.01 to about 0.5 weight percent, based on the total weight of the oral rinse composition. In various embodiments, a preservative can be present in an amount of at least about 0.01 weight percent, based on the total weight of the oral rinse composition (e.g., with a maximum concentration of about 5 weight percent).

In various embodiments, the oral rinse composition can further comprise one or more additional ingredients. For example, the oral rinse composition can comprise at least one of a sweetener and a flavoring agent. In some embodiments, a sweetener can be present in an amount in the range of about 0.01 to about 5 weight percent, or about 0.1 to about 1 weight percent, or about 0.2 to about 0.5 weight percent, based on the total weight of the oral rinse composition. In various embodiments, a sweetener can be present in an amount of at least about 0.01, or at least about 0.1 weight percent, based on the total weight of the oral rinse composition (e.g., with a maximum concentration of about 5 weight percent). In some embodiments, a flavoring agent can be present in an amount in the range of about 0.01 to about 5, or about 0.1 to about 2 weight percent, or about 0.5 to about 1 weight percent, based on the total weight of the oral rinse composition. The flavoring agent can be present in an amount of at least about 0.01 weight percent, or at least about 0.1 weight percent, or at least about 0.5 weight percent, based on the total weigh of the oral rinse composition (e.g., with a maximum concentration of about 5 weight percent).

In one aspect of the present invention, a method of forming an oral rinse composition is provided. Said method can be particularly useful for avoiding the undesirable formation of one or more precipitates. In particular, the method can be useful for completely avoiding the formation of one or both of zinc oxychloride and zinc phosphate precipitates, or avoiding forming more than only a trace amount of said precipitates, as already defined herein. In one or more embodiments, the method can comprise forming a solution of zinc chloride in water, adding the zinc chloride solution to a mixture of at least one sugar alcohol and water to form a premix solution, and mixing at least one pyrophosphate into the premix solution until the at least one pyrophosphate is fully dissolved and an intermediate solution is formed. In certain embodiments, additional ingredients can be mixed into the intermediate solution to provide a final oral rinse composition.

In various embodiments of the present invention, the method can comprise first making a premix solution. The premix solution can comprise zinc chloride, water, and at least one sugar alcohol. As discussed above, before adding the zinc chloride to the premix solution, a zinc concentrate or a concentrated solution of zinc chloride in deionized water can be prepared or provided. In various embodiments, the zinc chloride solution can be at least about 40% zinc chloride in water, at least about 50% zinc chloride in water, or at least about 60% zinc chloride in water. In certain embodiments, the zinc chloride solution can be about 40% to about 75%, or about 45% to about 70%, or about 50% to about 65% zinc chloride in water. As discussed above, zinc oxychloride typically forms in excess water. As such, the zinc chloride solution should not have excess water which would result in zinc oxychloride precipitating. In other words, the zinc chloride can be fully dissolved such that the zinc chloride solution is a clear liquid.

In certain embodiments, increasing the temperature of the water used to form the zinc chloride solution and/or heating the zinc chloride solution during mixing can assist with fully dissolving the zinc chloride. In certain embodiments, the water and/or the zinc chloride solution can be heated to a temperature of at least about 20° C., at least about 30° C., or at least about 40° C. prior to and/or during mixing of the zinc chloride solution.

Separately from the zinc chloride solution, a sugar alcohol solution comprising at least one sugar alcohol and water can be prepared. It is noted that additional water in the composition can assist with solubilizing the pyrophosphate(s) in the final oral rinse composition. However, as discussed above, adding the zinc chloride solution to additional water can cause zinc oxychloride to precipitate due to the excess water. It was surprisingly discovered that first forming or providing zinc chloride in the form of a zinc chloride concentrate (i.e., zinc chloride solution), and then adding the zinc chloride concentrate to the bulk of the water in the final oral rinse composition, wherein the bulk of the water comprises at least a relatively small amount of a sugar alcohol or a sugar alcohol solution, eliminates the precipitation of a zinc salt. As such, in order to avoid the precipitation of zinc oxychloride, at least one sugar alcohol can be mixed with deionized water until the at least one sugar alcohol is fully dissolved. The sugar alcohol solution and the remaining water of the oral rinse composition can then be mixed with the zinc chloride solution to form a premix solution.

After preparing the premix solution, at least one pyrophosphate can be added to the premix solution. The premix solution can be stirred until the at least one pyrophosphate is fully dissolved and an intermediate solution is formed. Without being limited by theory, forming the premix solution, which includes the zinc chloride solution, at least one sugar alcohol, and excess water, before adding the at least one pyrophosphate to the composition can help eliminate or at least reduce zinc oxychloride and zinc phosphate from precipitating in the oral rinse composition. The excess water in the premix solution can assist with dissolving the pyrophosphate(s) without zinc phosphate precipitating. After the pyrophosphate(s) is fully dissolved, additional ingredients, as discussed above in more detail, can be mixed into the intermediate solution if desired. It is noted that the additional ingredients are not limited to the specific ingredients discussed above.

In various embodiments, adjusting the pH and/or temperature of the premix solution prior to adding at least one pyrophosphate can assist with reducing or completely avoiding formation of one or more undesirable precipitates (e.g., zinc oxychloride and zinc phosphate). For example, lowering the pH of the premix solution prior to adding the at least one pyrophosphate can help counteract the increase in pH that can occur with the addition of the at least one pyrophosphate. In certain embodiments, the pH of the premix solution can be lowered to a pH of about 5 to about 7 prior to adding the at least one pyrophosphate.

Detailed methods of making embodiments of the oral rinse compositions disclosed herein are provided in the examples below.

### EXAMPLES

### Example 1

An embodiment of an oral rinse composition (referred to as “Formula 1”) is provided. Table 1 below provides ingredients included in an embodiment of the oral rinse composition of the present invention. The ingredients are generally listed by a preferred order of addition, as described in more detail below. Table 1 also includes the weight percentage of each ingredient, based on the total weight of the oral rinse composition.

TABLE 1
Oral Rinse Composition Formula 1

| Batch | Ingredient | Weight Percentage | |
|---|---|---|---|
| Premix | Zinc Chloride | 0.01-3 | wt. % |
| Premix | Water | 50-85 | wt. % |
| Premix | Sorbitol (70% Sorbitol Solution) | 15-40 | wt. % |
| Main | Methyl Salicylate | 0.01-2 | wt. % |
| Main | Tetrasodium Pyrophosphate (TSPP) | 0.1-3 | wt. % |
| Main | Disodium Pyrophosphate (DSPP) | 0.1-3 | wt. % |
| Main | Benzyl Alcohol | 0.01-2 | wt. % |
| Main | Ethyl Alcohol | 0.1-5 | wt. % |
| Main | Poloxamer | 0.1-5 | wt. % |
| Main | Polysorbate | 0.1-3 | wt. % |
| Main | Sodium Benzoate | 0.01-2 | wt. % |
| Main | Menthol | 0.01-2 | wt. % |
| Main | Sodium Saccharin | 0.01-2 | wt. % |

### Example 2

A method of making a full scale batch (800 KG) of the oral rinse composition provided in Example 1, Formula 1, is provided.

TSPP was fully dissolved in a mixture of the water and sorbitol. DSPP, saccharin, and zinc chloride were added simultaneously after TSPP was fully dissolved in a mixture of the water and sorbitol. After mixing, the remaining ingredients listed in Table 1 were added.

After the addition of DSPP, saccharin, and zinc chloride, there were white specks that did not fully go into solution after extensive mixing. The specks were isolated and identified as zinc phosphate. TSPP caused an increase in pH (9+) when added to a water/sorbitol mixture.

Without being limited by theory, the increase in high pH may have contributed to the formation of zinc phosphate.

### Example 3

Several methods of making the oral rinse composition provided in Example 1, Formula 1, on a pilot scale (15 KG) were tested.

With US sourced raw materials, deionized water and sorbitol were combined. TSPP was added and fully dissolved. Zinc chloride was then added. This resulted in clear solution after 20 minutes of mixing.

With US sourced raw materials, deionized water and sorbitol were combined. TSPP and zinc chloride were then simultaneously added and fully dissolved. This resulted in clear solution after 30 minutes of mixing.

With Montreal sourced raw materials, deionized water and sorbitol were combined. TSPP was added and fully dissolved. Zinc chloride was then added. This resulted in clear solution after 20 minutes of mixing.

With Montreal sourced raw materials, deionized water and sorbitol were combined. TSPP and zinc chloride were then simultaneously added and fully dissolved. This resulted in clear solution after 30 minutes of mixing.

With Montreal sourced raw materials, deionized water and sorbitol were combined. Zinc chloride, TSPP, DSPP, and saccharin were then simultaneously added and mixed. This resulted in clear solution after 40 minutes of mixing.

With Montreal sourced raw materials, deionized water and sorbitol were combined. TSPP was added and fully dissolved. Zinc chloride, DSPP, and saccharin were then simultaneously added and mixed. This resulted in clear solution after 40 minutes of mixing.

The insoluble zinc phosphate precipitate did not form on the small scale trials. However, the trials indicated that fully dissolving the TSPP, DSPP, and saccharin prior to the addition of zinc chloride resulted in faster dissolution of zinc chloride. However, it was determined that this method was not as effective (in the sense that additional manipulation of materials was required) as first forming a zinc chloride concentrate which was combined with the remaining water of the composition, wherein the remaining water comprised at least a small amount of sorbitol or a sorbitol solution. This indicated that pre-dissolving zinc chloride can be beneficial.

### Example 4

A method of making a full scale batch (800 KG) of the oral rinse composition provided in Example 1, Formula 1, is provided.

A 50% zinc chloride solution was made and then diluted with deionized water, but a zinc oxychloride precipitate immediately formed. It was determined that once the 50% zinc chloride solution was added to the tank, the container should not be rinsed to avoid creating the precipitate.

Water and sorbitol were added to the main mix tank followed by TSPP and mixed until fully dissolved. Then DSPP and saccharin were added into the main mix tank and mixed until fully dissolved. Then the zinc chloride solution was added to the main mix tank. A precipitate that looked like zinc phosphate appeared but eventually dissolved and went into solution.

### Example 5

Several methods of making the oral rinse composition provided in Example 1, Formula 1, on a pilot scale were tested to optimize the process of making the 50% zinc chloride solution and to identify how the mixing container could be rinsed out without producing the zinc oxychloride precipitate.

A base solution was made with deionized water, sorbitol, TSPP, DSPP, and sodium saccharin.

A 50% zinc chloride solution was made and then added to the base solution while still hot. A cloudy, white precipitate formed, then cleared.

A 35% zinc chloride solution was made and then added to the base solution while still hot. A cloudy, white precipitate formed, then cleared.

A 50% zinc chloride solution was made, cooled to room temperature, and then added to the base solution. A cloudy, white precipitate formed, then cleared.

A 35% zinc chloride solution was made, cooled to room temperature, and then added to the base solution. A cloudy, white precipitate formed, then cleared.

Charged deionized water, sorbitol, and saccharin were mixed until dissolved. A 50% zinc chloride solution was added and mixed. The solution was clear. TSPP was added and mixed. The solution was slightly cloudy, but cleared with specks still present. After 50 minutes of mixing, the solution was clear. Charged DSPP was added. The solution was slightly cloudy, but cleared quickly. This pilot trial was scaled up to a full batch by adding sodium benzoate, poloxamer, polysorbate, benzyl alcohol, blue dye soln., and alcohol premix (menthol, methyl salicylate). The end result was a clear rinse. This order of addition of the ingredients was repeated as a full pilot batch (150 KG). It was necessary to preheat the water when mixing the zinc chloride solution to get it fully dissolved. The mix time to get the TSPP into solution was 28 minutes.

It was determined that addition additional water to rinse out a container that previously contained zinc chloride solution resulted in zinc oxychloride precipitate. A trial was conducted to determine if hotter water helped with the zinc chloride solution rinse of the mixing container. Water at 22° C. immediately formed precipitate. Water at 50° C. formed only a slight precipitate.

A pilot trial was conducted to determine how sorbitol was affecting precipitate. A deionized water/sorbitol solution was made up and added to an empty chamber. No precipitate formed, but solution was slightly cloudy. A second trial was done with sorbitol as a rinse first, and then water was added. No precipitate formed. It was confirmed that even a small amount of sorbitol present in the bulk of the water of the oral rinse composition eliminated the zinc oxychloride precipitate.

A trial was conducted to verify that adding a hot zinc chloride solution to the batch would not have a negative effect. Deionized water was heated, zinc chloride was added and mixed. The solution was 60° C. and immediately added to a sorbitol/deionized water solution. The solution remained clear.

### Example 6

A method of making a full scale batch (800 KG) of the oral rinse composition provided in Example 1, Formula 1, is provided.

A 50% zinc chloride solution was prepared. Water and sorbitol were added to the main mix tank followed by saccharin. Saccharin was allowed to fully dissolve before adding the zinc chloride premix. The premix was added through the top of the tank but two smaller size zinc chloride premix solutions were made up to eliminate the safety concern of the container weight. it was decided this was the better option than to pump in and chase with sorbitol based on the logistics of weighing up the sorbitol needed. The batch stayed completely clear upon charging the zinc chloride premix. TSPP was then added and it took 70 minutes to fully dissolve. Small specks appeared during mixing. It was not clear whether the specks were TSPP or zinc phosphate but they eventually dissolved. The remainder of the raw materials were added without issue.

### Example 7

A method of making the oral rinse composition provided in Example 1, Formula 1, is provided.

Pilot trials (25 KG and 150 KG) were conducted using a 62% zinc chloride solution in the method provided in Example 6 above. There were no precipitation issues. Mix times for TSPP to go into solution were 30 minutes (25 KG) & 23 minutes (150 KG). These pilot trails were repeated as a full scale batch (8000 KG). Again, a 62% zinc chloride solution was used in the method provided in Example 6 above. To improve on batch time and dissolution of TSPP, it was discovered that sifting TSPP powder through a screen to reduce clumps in the TSPP resulted in a more expedient dissolution, about 30 minutes, which is equal or faster than dissolution in earlier batches when TSPP was added in the mix before the zinc chloride solution was added. Although the dissolution time of the TSPP was significantly reduced, it was not determined if it was because of the zinc chloride solution or the sifting of the TSPP.

Many modifications and other embodiments of the disclosure will come to mind to one skilled in the art to which this disclosure pertains having the benefit of the teachings presented in the foregoing description; and it will be apparent to those skilled in the art that variations and modifications of the present disclosure can be made without departing from the scope or spirit of the disclosure. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. An oral rinse composition comprising:
zinc chloride;
water, wherein the amount of water present in the composition is at least about 80%, based on the total weight of the composition;
one or more sugar alcohols; and
optionally, one or more pyrophosphates;
wherein the oral rinse composition is substantially free of one or both of zinc oxychloride and zinc phosphate.

2. The oral rinse composition of claim 1, wherein one or more of the following conditions is met:
the zinc chloride is present in an amount in the range of about 0.01 to about 5 weight percent, based on the total weight of the oral rinse composition;
the one or more pyrophosphates are present in amounts in the range of about 0.01 to about 5 weight percent, based on the total weight of the oral rinse composition;
the one or more sugar alcohols are present in amounts in the range of about 15 to about 35 weight percent, based on the total weight of the oral rinse composition.

3. The oral rinse composition of claim 1, wherein the one or more pyrophosphates comprise at least one of tetrasodium pyrophosphate and disodium pyrophosphate.

4. The oral rinse composition of claim 1, wherein the one or more sugar alcohols comprise sorbitol.

5. A method of making an oral rinse composition comprising:
providing a zinc chloride solution;
forming a premix comprising the zinc chloride solution, one or more sugar alcohols, and deionized water;
optionally mixing the premix with one or more pyrophosphates until the pyrophosphates are dissolved in an intermediate solution; and
mixing additional ingredients into the intermediate solution to form the oral rinse composition;
wherein the oral rinse composition is substantially free of zinc oxychloride and zinc phosphate.

6. The method of claim 5, wherein the zinc chloride solution is a 50% zinc chloride in water solution.

7. The method of claim 5, further comprising adjusting the pH of the premix to a pH in the range of about 5-7.

8. The method of claim 5, further comprising:
heating water to an increased temperature; and
mixing zinc chloride with the heated water to form the zinc chloride solution.

9. The method of claim 8, wherein the increased temperature is at least about 30° C.

10. The method of claim 5, wherein the one or more pyrophosphates comprise at least one of tetrasodium pyrophosphate and disodium pyrophosphate.

11. The method of claim 5, wherein the one or more sugar alcohols comprise sorbitol.

12. The method of claim 5, wherein one or more of the following conditions is met:
the zinc chloride is present in an amount in the range of about 0.01 to about 5 weight percent, based on the total weight of the oral rinse composition;
the one or more pyrophosphates are present in amounts in the range of about 0.01 to about 5 weight percent, based on the total weight of the oral rinse composition;
the one or more sugar alcohols are present in amounts in the range of about 15 to about 35 weight percent, based on the total weight of the oral rinse composition.

13. The method of claim 5, wherein the oral rinse composition is manufactured in the form of a final oral rinse product having a desired dosage size, shape and weight.
